(19) United States
(12) Patent Application Publication
(10) Pub. No.: US 2004/0001898 A1
(43) Pub. Date: Jan. 1, 2004

(54) Compositions and methods for detoxification and cancer prevention

(76) Inventors: Armand Malnoe, Montaubion-Chardonney (CH); Christophe Cavin, Poliez-Pittet (CH); Elizabeth Offord-Cavin, Poliez-Pittet (CH)

Correspondence Address: BELL, BOYD & LLOYD LLC, P. O. BOX 1135, CHICAGO, IL, 60690-1135, US

(21) Appl. No.: 10/180,773

(22) Filed: Jun. 26, 2002

(51) Int. Cl. A61K 35/78

(52) U.S. Cl. 424/764; 424/725

(57) ABSTRACT

Nutritional compositions capable of reducing the risk of cancer are provided. The nutritional compositions combine the added effects of both a prebiotic source and a phytochemical(s) capable of inducing enzymatic activity in mammals to reduce the incidence of cancer. The prebiotic and phytochemical source can be derived from a single plant material, such as chicory.

### BACKGROUND OF THE INVENTION

The present invention generally relates to nutritional compositions. More specifically, the present invention relates to nutritional compositions that include plant material, such as chicory and/or extracts thereof, to enhance health in humans and animals.

The need to enhance health in mammals has involved and continues to involve on-going research efforts and discoveries to prevent disease. In general, food, dietary or other nutritional sources are known to contain a number of constituents or agents which are believed to be capable of protecting against disease in humans and animals.

For example, oligosaccharides, such as inulin and various fructo-oligosaccharides, are reported to have prebiotic effects, such as promoting the growth of bifido- and lacto-bacteria in the gastro-intestinal tract at the expense of pathogens including, for example, Clostridium perfringens. See, for example, Gibson et al., Food Microbiology, 11 (6), pp. 491-498 (1994). Although most reported experimentation has been carried out in vitro, there have been reports that these oligosaccharides have a similar effect in the gut of rats and humans. In this regard, it is generally known that the promotion of growth of bifido- and lacto-bacteria through the use of oligosaccharides can provide a variety of different beneficial effects on animals and humans, such as the prevention and/or treatment of diarrhea, increased growth, improved ability to breed or other like beneficial effects which enhance health.

Further, a primary mechanism by which dietary agents are believed to protect against cancer is by inducing enzymatic detoxification to prevent cancer causing agents from reacting or binding with critical target sites susceptible to cancerous cell growth. Research has indicated that bioactive compounds, such as cafestol and kahweol in coffee, can induce the total activity of glutathione-S-tranferase (“GST”.) A. C. Huggett et al.; Chemoprotective Effects Of Coffee And Its Components Cafestol And Kahweol: Effects On Xenobiotic Metabolising Enzymes, Asic, 16 Colloque, Kyoto, pp. 65-72 (1995). The GST enzymes are generally believed to be an essential family of enzymes with tissue-specific distribution that are suitable for detoxification and thus cancer prevention. It is generally known that such detoxification enzymes can promote carcinogen inactivation by their ability to catalyse the reaction of a wide variety of electrophiles to glutathione. In this regard, it is generally believed that most activated carcinogens are electrophiles. Wim A. Nijhoff et al.; Effects Of Consumption Of Brussel Sprouts On Plasma And Urinary Glutathione-S-Tranferase Class-α And-π In Humans; Carcinogenesis, vol. 16 no. 4, pp. 955-957 (1995).

Inulin or other dietary agents that are believed to promote health in humans and animals as discussed above, in general, can be derived from plants or other natural sources. For example, inulin is generally known to be purified from plants which contain high concentrations of inulin, such as chicory, Jerusalem artichoke, leek and asparagus. In this regard, the plant is typically purified or otherwise treated prior to use in order to enhance a plant's flavor, such as to eliminate, or at least minimize, a bitter flavor typically associated with chicory. See, for example, U.S. Pat. No. 4,865,852. In chicory, the bitter flavor is believed to be derived from sesquiterpene lactones, such as lactucin and lactucopicrin. Further, it is believed that purification or other treatment of the plant prior to use can provide more accurate control of the amounts of bioactive agents, such as inulin, to be added to the diet of a human and/or animal.

In general, the purified plant product is prepared by hydrolysis with acids or enzymes. The hydrolysate is then collected and condensed to obtain the bioactive agent, such as inulin. For example, Japanese Patent Document No. 63-309147 discloses grinding chicory tubers, partially hydrolyzing them with acids, and then drying the hydrolysate with or without neutralization. However, the purification of, for example, fructo-oligosaccharides and inulin, can greatly add to the cost of the dietary product. Consequently, the use of such dietary products has been generally limited to specialty food or dietary products in humans and animals.

A need, therefore, exists for a nutritional composition that includes natural ingredients, such as chicory and/or extracts thereof, that are palatable to humans and animals, that can be inexpensively produced, and that can enhance health in humans and animals, such as the prevention of cancer.

### SUMMARY OF THE INVENTION

The present invention relates to nutritional compositions that can be utilized to enhance health in humans and animals, particularly the prevention of cancer. The nutritional compositions of the present invention at least include a combination of fermentable dietary fibers, preferably prebiotic fibers ( e.g., inulin or the like) and phytochemical agents.

Applicants have discovered that the combination of fermentable dietary fiber, preferably prebiotic fibers, and phytochemical agents can be derived from a common source, such as a plant source including chicory. Using in vitro models of rodent hepatocytes, Applicants have demonstrated that chicory root extracts also contain phytochemicals (in addition to fermentable dietary fiber, preferably prebiotic fibers, such as inulin). The phytochemicals are capable of promoting enzyme activity, such as GST enzyme activity, which can promote detoxification, stimulate an antioxidant defense or promote other like processes in a mammal which are believed to be responsible for the prevention of cancer. In this regard, Applicants have surprisingly found that the combination of prebiotic fibers (e.g., inulin), which have known anti-tumor properties, and chemopreventive phytochemicals can both originate from the same plant source that can be utilized to reduce the risk of cancer, particularly cancer associated with the gut or other tumor-sensitive sites.

To this end, in an embodiment of the present invention, a nutritional composition capable of reducing a risk of cancer is provided. The nutritional composition can include any natural or purified fermentable dietary fiber, preferably prebiotic fiber, and a phytochemical agent capable of inducing an enzyme activity in a mammal. Preferably, the plant material in the nutritional composition includes an amount of at least 0.5% by weight on a dry weight basis.

In an embodiment, the plant material can include chicory, Jerusalem artichoke, leek, asparagus, extracts thereof, roots thereof, pulp thereof, the like and combinations thereof. In embodiment, the plant material includes a chicory including, for example, chicory extract, chicory pulp, chicory root, the like and combinations thereof.

In an embodiment, the enzyme activity is conducted via glutathione-S-transferase.

In an embodiment, the enzyme activity is capable of promoting detoxification in tissue of the mammal. In another embodiment, the enzyme activity is capable of stimulating an antioxidant defense in tissue of the mammal.

The present invention also provides a pet food product. The pet food product includes a starch matrix; and a biologically effective amount of a plant material including a source of fermentable dietary fiber, preferably prebiotic fiber, and a phytochemical agent capable of inducing an enzymatic activity in a mammal to reduce the risk of cancer.

In still another embodiment of the present invention, a method of preparing a nutritional food product capable of reducing incidence of cancer in a mammal is provided. The process includes the steps of providing a plant material; processing the plant material to form a plant extract including a source of fermentable dietary fiber and a phytochemical agent capable of inducing enzyme activity in the mammal; and processing the plant extract and one or more food ingredients to form the nutritional food product that includes at least 0.5% by weight of the plant extract.

In an embodiment of the present invention, the plant extract is processed by defatting the plant material to form a first plant extract and subsequently processing the first plant extract with ethyl acetate via acid hydrolysis to form the plant extract.

In a further embodiment of the present invention, a method of reducing a risk of cancer in a mammal at risk of cancer is provided. The method includes the steps of administering to the mammal a biologically effective amount of a nutritional composition that contains a natural or purified fermentable dietary fiber, preferably prebiotic fiber and a phytochemical agent capable of stimulating enzyme activity in the mammal. Preferably, the nutritional composition contains about 0.5% to about 2% by weight of the plant material selected from the group consisting of chicory, Jerusalem artichoke, leek, asparagus, extract thereof, pulp thereof, root thereof, the like and combinations thereof.

In yet a further embodiment of the present invention, a method of increasing detoxification in tissue of a mammal is provided. The method includes the steps of administering to the mammal a nutritionally complete food product including a plant material that contains a source of fermentable dietary fiber, preferably prebiotic fiber and a phytochemical agent capable of inducing an enzymatic activity in the mammal. It is believed that the increased enzymatic activity can promote detoxification thereby reducing a risk of incidence of cancer.

In still yet a further embodiment of the present invention, a method of stimulating an antioxidant defense in tissue of a mammal is provided. The method includes administering to the mammal a biologically effective amount of a composition including a plant material that contains a source of fermentable dietary fiber, preferably prebiotic fiber and a phytochemical agent capable of inducing an enzymatic activity in the mammal. It is believed that the increased enzymatic activity can stimulate the antioxidant defense thereby reducing a risk of incidence of cancer.

An advantage of the present invention is to provide an improved nutritional composition that can be utilized to reduce an incidence of cancer in mammals. In this regard, the nutritional composition is capable of promoting detoxification processes, antioxidant defenses, other like processes or combinations thereof in mammals which are believed to reduce an incidence of cancer.

Another advantage of the present invention is to provide an improved nutritional composition that includes a plant material containing a source of fermentable dietary fiber, preferably prebiotic fiber and a phytochemical(s) capable of inducing an enzymatic activity in order to reduce the risk of cancer in mammals at risk of cancer.

Yet another advantage of the present invention is to provide methods of producing improved nutritional compositions containing a plant material that can enhance the palatability of the nutritional composition while maintaining the enzymatic detoxification properties of the plant material.

A further advantage of the present invention is to provide methods of prevention against cancer in mammals that include the administration of an improved nutritional composition. The nutritional composition contains both a source of fermentable dietary fiber, preferably prebiotic fiber, and phytochemical agents such that the combined effect of same can be utilized to reduce the risk of cancer.

Additional features and advantages of the present invention are described in, and will be apparent from, the following Detailed Description of the Invention.

### DETAILED DESCRIPTION OF THE INVENTION

The present invention relates to nutritional compositions that at least include a source of fermentable dietary fiber and phytochemical agents derived from natural sources, such as a plant material which can be utilized to effectively prevent cancer in humans and animals, particularly cancer associated with the gut, including, for example, colon cancer. Applicants have surprisingly discovered that certain plants and/or plant extracts thereof, such as chicory, contain phytochemicals which can stimulate detoxification pathways to prevent cancer, such as stimulate phase II enzymes including, for example, glutathione-S-transferase or the like.

In addition to phytochemicals as discussed above, plants, such as chicory, are known to contain fermentable dietary fibers, such as prebiotic fibers. The prebiotic fibers can include a number of suitable materials, such as oligosaccharides including inulin. The fermentable dietary fibers are also believed to reduce cancer incidence, particularly in the colon. In this regard, Applicants believe that enhanced benefits with respect to cancer prevention in humans and animals can be realized due to the combined effect of fermentable dietary fibersand phytochemicals that can stimulate detoxification pathways.

Applicants have also demonstrated that the detoxifying properties of the nutritional composition of the present invention are essentially unaffected by the processing conditions under which the nutritional compositions are prepared pursuant to present invention. For example, purification of the plant material made pursuant to the present invention which can be utilized to reduce the bitterness of the plant extract and thus enhance human and animal palatability has negligible, if any, effect on the detoxification properties of the resultant purified product. In this regard, the desirable cancer prevention properties can result from essentially crude plant extracts, such as chicory. This can eliminate the need for expensive purification or other like treatment of a plant material to produce the desirable bioactive fraction(s).

As used herein, the term “bioactive agent” or other like terms, such as “bioactive fractions”, means any constituent or constituents that can display biological activity, chemical activity or like activity in a mammal(s) that are capable of enhancing health in a mammal. Examples of bioactive agents include, for example, fermentable dietary fibers, such as prebiotic fibers, phytochemicals or the like.

As used herein, the term “prebiotic” or other like terms including “prebiotic fiber” means a substance or a constituent that can promote the growth of beneficial microorganisms in mammals.

As used herein, the term “phytochemical” or other like terms including “phytochemicals” and “phytochemical agent” means any chemical produced by a plant that is believed to impact health benefits to humans and/or animals, such as the prevention of cancer.

As used herein, the term “enzymatic activity” or other like terms, such as “enzyme activity”, means any suitable enzyme which can act as a catalyst during any suitable biological, chemical or other like process which is believed to effect health in a mammal. For example, the increase of enzyme activity relating to glutathione-S-transferase can promote detoxification in tissue, stimulate an antioxidant defense in tissue or like processes which are believed to be responsible for the prevention of cancer.

The nutritional composition can include any suitable and compatible types and amounts of constituents such that the nutritional composition can be effectively utilized to prevent cancer. In an embodiment, the nutritional composition includes a plant material that contains one or more fermentable dietary fibers, preferably prebiotic fibers, and phytochemical agents which are capable of inducing or promoting enzyme activity, such as GST enzyme activity, that is believed to be responsible for the detoxification of cancer causing agents. In addition to promoting detoxification via increased enzymatic activity, the phytochemical agent is also believed to stimulate antioxidant defenses, or stimulate other like processes in humans and/or mammals. As a result, the phytochemical agent is believed to be capable of enhancing health in the mammal, such as reducing the incidence of cancer. A number of plant materials can be effectively added to the nutritional composition including, for example, chicory, Jerusalem artichoke, leek, asparagus, extracts thereof, pulps thereof, roots thereof, the like and combinations thereof. In an embodiment, chicory, chicory extract, chicory pulp, chicory root, the like and combinations thereof are preferred.

In an embodiment, the plant material is added to the nutritional composition in the form of an extract, such as a chicory extract. The plant extract can be made from any suitable part of the plant material includes, for example, the root, the pulp, the like or combinations thereof. The extract is processed such that its flavor can be enhanced. For example, bitter flavors which are typically associated with plant materials, such as chicory, can be removed by processing the plant into an extract. The extract can also be prepared such that the amount of bioactive agent in the final extract product can be desirably controlled.

It should be appreciated that the plant material can be processed to form an extract in a variety of different and suitable ways. In general, the plant material, such as the chicory root, is ground, powdered or provided in any suitable form. The plant material can then be further processed in a number of different stages to produce the product extract. In an embodiment, a defatting procedure is performed on the plant material to produce an extract that results from fats removed from the plant material. The defatting procedure can be conducted under any suitable defatting process conditions with any suitable types and amounts of solvents including, for example, hexane.

In an embodiment, the resultant extract of the defatting procedure can be further processed via acid hydrolysis to produce another type of plant extract that can be added to the nutritional composition of the present invention. The acid hydrolysis procedure can be conducted under any suitable process condition with any suitable types and amounts of solvents, including, for example, ethyl acetate.

In an embodiment, the extract from the defatting procedure can be further processed via a solvent extraction procedure. The solvent extraction can be carried out under any suitable process conditions and in the presence of any suitable amount and type of solvent. In an embodiment, the solvent includes a solution of methanol (“MeOH”) and water mixed in a 1:1 volume ratio. The resultant solution of the solvent extraction procedure can be further processed by evaporation of the solvent under suitable conditions to produce another extract. Alternatively, the resultant solution can be treated with an adsorbant agent, such as polyvinylpolypyrrolidone or the like, to trap polyphenols. The adsorbant agent treatment can be carried out under any suitable process conditions. Specific examples of preparing plant extracts in accordance with an embodiment of the present invention are detailed below.

In an embodiment, the fermentable dietary fiber(s) and phytochemical agent(s) of the nutritional composition can be derived from a common or the same plant material, such as chicory. As previously discussed, Applicants believe that the combined effect of the fermentable dietary fiber, preferably prebiotic fiber, and phytochemical source of plants, such as chicory, can result in an enhanced chemoprotective effect such that cancer in mammals can be prevented. The prebiotic fiber can include any suitable amount and type including, for example, oligosaccharides, such as inulin and various fructo-oligosaccharides, soy oligosaccharides and combinations thereof.

The phytochemical agents can include any suitable type and amount such that it is capable of inducing enzyme activity that is believed to be responsible for the detoxification of carcinogens. In an embodiment, the phytochemical agent includes antioxidants, cafestol, kahweol, catechin, like constituents or combinations thereof In an embodiment, the phytochemical agent of the plant material is capable of inducing phase II enzyme activity, such as GST enzyme activity. By inducing enzyme activity, such as GST activity, the phytochemicals of the present invention are believed to be capable of increasing detoxification in mammal tissues (e.g., increasing GST activity), capable of stimulating an antioxidant defense in mammal tissues (e.g., increasing levels of glutathione), or capable of enhancing health in mammals in other like mechanisms which are believed to result in, for example, the prevention of cancer.

It should be appreciated that the nutritional composition of the present invention can include a variety of different and suitable forms. In an embodiment, the nutritional composition can include a nutritional supplement, a food preparation for humans and/or animals, pet food or the like. The nutritional composition can be added to the food product in any suitable amount. In an embodiment, the food product includes the plant material of the nutritional composition in an amount of at least 0.5% by weight, preferably from about 0.5% to about 30% by weight, more preferably about 0.5% to about 2% by weight.

In an embodiment, the present invention provides a pet food product that includes a starch matrix and an effective amount of a plant material wherein the plant material includes a source of fermentable dietary fiber, more preferably a prebiotic fiber, and phytochemical agent capable of inducing enzymes or enzyme activity which is believed to enhance health in mammals, such as to prevent cancer as previously discussed. The pet food product of the present invention can include any suitable number, type and amount of constituents and be processed in any suitable way to form a desirable product form.

In an embodiment, the present invention includes a gelatinized cereal product which contains an amount of a plant material. The plant material at least includes a source of fermentable dietary fiber and phytochemicals capable of stimulating enzymatic activity in mammals which is believed to enhance health in the mammal as described above.

In an embodiment, the plant includes inulin in an amount sufficient to provide at least about 0.25% by weight inulin, on a dry matter basis. The plant material used may be any suitable source, for example, chicory, Jerusalem artichoke, leek, onion, yacon, asparagus, which contains high levels of inulin, extracts thereof, pulp thereof, root thereof and mixtures of these plants. In an embodiment, chicory and Jerusalem artichoke are preferred. In an embodiment, the plant materials include at least 50% by weight of inulin. For ease of handling, the plant material is preferably in a dried and comminuted or powder form. As described below, the processes utilize dried, comminuted chicory and/or extracts thereof However, it is to be understood that any suitable plant material may be used in any suitable form and added to the cereal product in any suitable amount.

As described below, the remaining ingredients included in the gelatinized cereal product may be any suitable ingredients commonly used in gelatinized cereal products. In general, these ingredients include a starch source and a protein source. Suitable starch sources are, for example, grains such as corn, rice, wheat, beets, barley, oats, soy, and mixtures thereof. Suitable protein sources may be selected from any suitable animal or vegetable protein source. Examples include meat meal, bone meal, fish meal, soy protein concentrates, milk proteins, gluten, and the like. The choice of the starch and protein sources will be largely determined by the nutritional needs of the animal or human, palatability considerations, the type of cereal product produced or other like considerations. Various other ingredients, for example, sugar, salt, spices, seasonings, vitamins, minerals, flavoring agents, fats and the like may also be incorporated into the gelatinized cereal product as desired.

The gelatinized cereal product may be produced in many different ways as desired. However, for a dried cereal product, an especially suitable way of producing the product is extrusion cooking. This may be done as is well known in the art. For example, in one suitable process, a feed mixture is fed into a preconditioner. The feed mixture is primarily made up of a starch source, a protein source, and the plant material, such as, chicory. In an embodiment, the chicory includes at least about 1% by weight of the feed material, preferably at least about 2% by weight. In an embodiment, the plant material ranges from about 10% to about 20% by weight, preferably about 10% by weight.

In the preconditioner, water or steam, or both, is mixed into the feed mixture. A sufficient amount of water or steam is mixed into the feed mixture to moisten the feed mixture. If desired, the temperature of the feed mixture may be raised in the preconditioner to about 60° C. to about 90° C. by weight. A suitable preconditioner is described in U.S. Pat. No. 4,752,139 herein incorporated by reference. It should be appreciated that the use a preconditioner is not required.

The moistened feed leaving the preconditioner is then fed into an extruder. The extruder may be any suitable single or twin screw and cooking extruder. Suitable extruders may be obtained from Wenger Manufacturing Inc., Clextral S A, Bühler A G, and the like. During passage through the extruder, the moistened feed passes through a cooking zone, in which it is subjected to mechanical shear and is heated. In an embodiment, the moistened feed is heated up to a maximum temperature of up to about 150° C., and a forming zone. The gauge pressure in the forming zone is about 300 kPa to about 10 MPa as desired. If desired, water or steam, or both, may be introduced into the cooking zone. During passage through the extruder, the starch source of the moistened feed is gelatinized to provide a gelatinized matrix structure primarily of starch, protein and the plant material, such as chicory.

The gelatinized matrix leaving the extruder is forced through a suitable die for example, a die as described in European Patent Application No. 0665051, the disclosure of which is herein incorporated by reference. A shaped extrudate, which has a cross-sectional shape corresponding to that of the orifice of the die, leaves the die. Depending upon the conditions in the extruder and the starch source used, the shaped extrudate expands to a greater or lesser extent. The shaped extrudate is then cut into pieces using blades. The individual pieces are then dried and, if desired, coated with protective or flavoring agents, or both. After cooling, the pieces may be packed into suitable packages. Alternatively, the individual pieces may be formed into flakes and then dried.

Depending upon the ingredients used, the gelatinized cereal product may be in the form of dried kibbles suitable for use as pet foods, expanded pieces suitable for use in breakfast cereals, flakes suitable for use in breakfast cereals, and the like.

It is also possible to produce a dried cereal product by mixing together water and the ingredients of cereal product, for example, by mixing in a preconditioner. The wet mixture may then be shaped into a desired shape by using, for example, shaping rollers. The shaped mixture may then be baked in an oven, at any suitable temperature. In an embodiment, the temperature ranges from about 220° C. to about 280° C. for a suitable baking time. In an embodiment, the baking time ranges from about 10 minutes to about 1 hour. The dried cereal product has the appearance of a baked biscuit.

If it is desired to produce a simulated meat product which may be used in canned pet foods, any suitable process can be used. For example, the processes can include those described in U.S. Pat. Nos. 4,781,939 and 5,132,137 which are herein incorporated by reference. In these processes, a protein source, especially a meat material, is emulsified. The meat material may be any suitable source of animal protein including for example, the muscular or skeletal meat of mammals, poultry, and fish or meat by-products, such as hearts, liver, kidneys, tongue and the like, or meat meals. Vegetable protein sources may also be included if desired. The exact composition may be selected according to cost and the desired flavor. The emulsification may be carried out in any suitable equipment.

The dried chicory is added to the emulsion. Also, if desired or needed, additional protein may be added to the emulsion. The additional protein may be any protein source as mentioned above. The exact choice will depend upon availability, cost and palatability. The additional protein can be added in any suitable amount. In an embodiment, the additional protein can be added in an amount ranging from about 5% to about 35% by weight.

If desired or required, fats may also be added to the emulsion. Usually the amount of fat in the emulsion must be controlled to facilitate processing and to obtain an acceptable product. However, the meat material may well contain the desired amount of fats and hence adjustment may not be necessary. Typically, at this stage the emulsion contains a maximum fat level of about 25% by weight. In an embodiment, the amount of fat in the emulsion is in the range of about 5% to 15% by weight, more preferably about 7% to about 12% by weight. The mass ratio of protein to fat in the emulsion is preferably about 1:1 to about 7:1. If added, the fats may be any suitable animal fats, such as tallow, or may be vegetable fats.

Additional ingredients such as sugars, salts, spices, seasonings, flavoring agents, minerals, and the like may also be added to the emulsion. In an embodiment, the amount of additional ingredients used ranges from about 1% to about 5% by weight of the gelatinized cereal product.

Water may also be added to provide from about 45% to 80% by weight moisture in the emulsion. If sufficient moisture is present in the meat material, water need not be added.

Once mixed, the emulsion is preferably fed through a vacuum stuffer, or similar de-aeration apparatus, to de-aerate the emulsion. This removes air which may otherwise cause disruption of the formulated emulsion product and reduce its meat-like appearance.

The emulsion is then fed to an emulsion mill which subjects the emulsion to rapid mechanical heating and shearing. Any suitable emulsion mill may be used including, for example, the emulsion mill disclosed in U.S. Pat. No. 5,132,137 herein incorporated by reference. Other suitable emulsion mills are commercially available under the trade name of TRIGONAL and may be obtained from Siefer Machinenfabrik GmbH & Co KG. Bahnhofstrasse 114, Postfach 101008., Velbert 1, Germany.

The temperature of the emulsion can be raised to the desired coagulation temperature in the emulsion mill in a few seconds. In an embodiment, the coagulation temperature ranges from about 100° C. to about 120° C. In an embodiment, the temperature ranges from about 45° C. to about 75° C. as described in U.S. Pat. No. 5,132,137. In general, the mechanical energy generated in the emulsion mill will be sufficient to heat the emulsion to the desired temperature but this may be supplemented by the injection of superheated steam.

The heated emulsion leaving the emulsion mill can be transferred to a holding tube. In the holding tube, the heated emulsion coagulates while moving slowly along the holding tube. The residence time of the heated emulsion in the holding tube is sufficient for the emulsion to have coagulated into a firm emulsion product upon reaching the exit of the holding tube.

The firm emulsion product leaving the holding tube is then transferred to a cutter where it is cut into pieces, such as chunks, of size suitable for use in a pet food. The chunks have the appearance and texture of meat. The chunks may be subjected to flaking if desired. The chunks may also be formulated into a chunk-in-gravy type of product.

Other procedures for producing chunks are known and may be used, such as extruding a feed mixture, cooking the feed mixture in a steam oven, and the cutting of the cooked extrudate into chunks.

If it is desired to produce a canned pet food in the form of a meat loaf, a meat batter may be prepared by emulsifying a suitable meat material to produce a meat emulsion. The meat material may be any suitable meat source, for example, as described above. Suitable gelling agents including gums, such as kappacarrageenan, locust bean gum, guar gum, xanthan gum or the like, may be added to the meat emulsion. In an embodiment, no more than about 2% by weight of gum is used. The dried plant material, such as chicory is then added to the meat emulsion.

Additional ingredients such as sugars, salts, spices, seasonings, flavoring agents, minerals, and the like may also be added to the meat emulsion. The amount of additional ingredients used is preferably such that they make up about 0.25% to about 5% by weight of the meat batter.

Water may also be added the meat emulsion to provide from about 70% to about 85% by weight. If sufficient moisture is present in the meat material, water need not be added.

The meat emulsion is then heated to a temperature above about 65° C. in a mixer-cooker. Steam may be injected into the meat batter if desired. The heated meat emulsion is then again emulsified to provide a loaf batter and the loaf batter maintained at a temperature above about 60° C. until filling into cans.

It should be appreciated that the gelatinized cereal product may be produced by any suitable process and not only those described above. Other types of oligosaccharides may also be included in the gelatinized cereal product, such as fructo oligosaccharide and soy oligosaccharide. The soy oligosaccharides may be added in the form of soy meal or other suitable soy source.

The cereal products may be in any suitable form including; for example, dried, semi-wet and wet. However, the matrix that makes up the cereal product must be gelatinized in order to remove or destroy the sesquiterpene compounds that may be present in the plant material. It should be appreciated that the cereal product of the present invention can be made for human and/or animal consumption.

By way of example, and not limitation, examples of pet food products made pursuant to an embodiment of the present invention are illustrated below.

### EXAMPLE 1

Dried Pet Food

A feed mixture is made up of about 58% by weight of corn, about 6% by weight of corn gluten, about 23% by weight of meat and meal, dried chicory and salts, vitamins and minerals making up the remainder. The dried chicory is in the form of a chicory extract made pursuant to an embodiment of the present invention and added in an amount of about 5% or less.

The feed mixture is fed into a preconditioner and moistened. The moistened feed is then fed into an extruder-cooker and gelatinized. The gelatinized matrix as it leaves the extruder is forced through a die and extruded, thus forming an extrudate. The extrudate is cut into pieces suitable for feeding to dogs, dried at about 110° C. for about 20 minutes, and cooled to form pellets. It should be appreciated that part or a totality of the fat mix, or of the fat and oils used, can be added at a later stage, for example, as a coating.

The added chicory can enhance health in a mammal, such as preventing cancer by, for example, promoting and/or stimulating detoxification, antioxidant defenses or the like in tissue as previously discussed.

### EXAMPLE 2

Dried Pet Food

A dry pet food is prepared like the dried pet food of Example 1. It further includes an additional ingredient typically associated with enhancing the palatability of the dry pet food suited to cats.

The added chicory can enhance health in a mammal, such as preventing cancer by, for example, stimulating and/or promoting detoxification, antioxidant defenses or the like in tissue, as previously discussed.

### EXAMPLE 3

Dry Cat Food

A feed mixture includes about 58% by weight of corn, about 6% by weight of corn gluten, about 23% by weight of chicken meal, dried chicory, salts, vitamins and minerals that make up the remainder. The chicory is added in an amount of about 5% or less. As previously discussed, the added chicory can stimulate enzymatic activity which is believed to enhance health in the animal, such as preventing cancer by, for example, promoting and/or stimulating detoxification, antioxidant defenses or the like in tissue.

The feed mixture is fed into a preconditioner and moistened. The moistened feed is then fed into an extruder-cooker and gelatinized. The gelatinized matrix as it leaves the extruder is forced through a die and extruded, thus forming an extrudate. The extrudate is cut into pieces suitable for feeding to cats, dried at about 110° C. for about 20 minutes, and cooled to form pellets. At this stage, a lyophilized powder of one or more strains of the Lactobacillus species, such as Lactobacillus rhamnosus NCC2583 (CNCM 1-2449), Lactobacillus acidophilus NCC2628 (CNCM 1-2453) and Enterococcus faecium SF68 (NCIMB 10415), is applied to the pellets. A sufficient amount of the powder is applied to the pellets such that the corresponding dietary intake amount for the cat is from about 1.0E+07 to about 1.0E+9 cfu/day. In this regard, a portion of the powder is mixed into a first mass of pellets which are subsequently bagged. A second portion of the powder is measured and mixed with a lipid carrier which is then sprayed on to a second mass of pellets. The pellets are bagged after the coating has dried sufficiently at 50-60° C. for some minutes.

### EXAMPLE 4

Canned Pet Food and Supplement

A mixture is prepared from about 73% of poultry carcass, pig lungs and beef liver (ground), about 16% of wheat flour, about 2% of dyes, vitamins, and inorganic salts. This mixture is emulsified at 12° C. and extruded into the form of a pudding. Dried chicory in the form of an extract made pursuant to an embodiment of the present invention is added to the emulsion in an amount of about 5% or less. As previously discussed, the added chicory can enhance health in a mammal. The emulsion is then cooked at a temperature of 90° C. It is cooled to 30° C. and then cut into chunks. About 45% of the chunks are mixed with about 55% of a sauce that is prepared from about 98% of water, about 1% of dye and about 1% of guar gum. Tinplate cans are filled with the chunk and sauce mixture and sterilized at 125° C. for about 40 minutes.

As a probiotic supplement to be mixed with the pet-food before serving, additional packaging in sachet form with strains of the following Lactobacillus species are provided Lactobacillus rhamnosus NCC2583 (CNCM 1-2449), Lactobacillus acidophilus NCC2628 (CNCM 1-2453) or Enterococcus faecium SF68 (NCIMB 10415). The corresponding dietary intake of the supplement for the pet is from about 106-10¹² cfu/day, depending on the type of pet, e.g., a cat or a dog, and on the pet's physical factors, such as body mass. The supplement is packaged such that it is removably attached to the can, together with feeding directions.

By way of example, and not limitation, experimental testing as described in detail below was conducted to demonstrate the effectiveness of the present invention.

In Vitro Testing

The inventors have conducted a number of experimental tests to demonstrate the beneficial effects of the present invention. In general, rat primary hepatocyte cultures were prepared and treated with varying amounts of chicory extracts over a period of about 48 hours. Experiments were then conducted to determine cytotoxic effects and effects of inducing or stimulating enzymatic activity in mammals which is believed to be responsible for enhancing the health of the mammal, such as by preventing and/or treating cancer by, for example, promoting detoxification, antioxidant defenses or the like in tissue as previously discussed. The preparation and experimental testing procedures and results are discussed below in greater detail.

Preparation of Cell Culture

Primary isolated hepatocytes were obtained by perfusion of the liver of Sprague-Dawley rats (250 g) with a collagenase solution as described in, for example, Sidhu J. S. et al., Influence of extracellular matrix overlay on phenobarbital-mediated induction of CYP 2B1, 2B2 and 3A1 genes in primary adult rat hepatocyte culture; Archive of Biochemistry & Biophysic; 301, pp. 1-11 , 1993. Cell viability, estimated by Trypan Blue exclusion test, was found to range between about 90 to about 95%.

The cells were seeded at a density of 10⁶ cells/ml on 60 mm plastic tissue culture dishes in 3 ml of William's medium supplemented with 2 mM L-glutamine, 10 mM Hepes pH 7.4, ITS+, 15000U Penicillin/Streptomycin, 100 nM Dexamethasone and 5% Fetal bovine serum (Hi-clone). Hepatocytes were allowed to attach for two hours and then washed with EBSS to remove debris and unattached cells. Fresh serum-free medium containing 25 nM of dexamethasone was added and an overlay of matrigel (233 μg/ml) was then applied. Fresh matrigel was added to the cultures every two days following medium change. To study the effects of chicory extracts made pursuant to an embodiment of the present invention, the components were added to culture media 24 hours after cell seeding over a period of 48 hours.

Preparation of Chicory Extracts

Four different chicory extracts, namely Extracts A-D, were prepared pursuant to an embodiment of the present invention. Initially, a 40 gram (g) and a 10 g sample of chicory ground to powder form were each sieved at 0.5 mm. The samples were then processed to remove fats by mixing the samples with hexane for about thirty minutes at room temperature. 600 milliliters (ml) of hexane was added to the 40 g chicory sieved sample, and 150 ml were added to the 10 g-sieved sample. The hexane was evaporated under vacuum at about 50° C. to form Extract A.

Extract B was prepared by first defatting 40 g of ground chicory powder as previously discussed. The defatted sample was hydrolyzed in 300 ml of an acid, such as HCl, in a boiling water bath for about 20 minutes. After cooling and centrifugation (8000 rpm, 5 minutes, 10° C.), the solution was extracted with 150 ml of a solvent, such as ethyl acetate, which is commercially available, such as from MERCK. The solvent is evaporated to dryness. After further drying on anhydrous sodium sulfate under vacuum at about 50° C., Extract B was formed.

Extracts C and D were prepared as follows. First, a 10 g sample of ground chicory powder was defatted as previously discussed. The second part of the defatted powder is extracted with about 250 ml of a solvent/water mix, such as a 1:1 volume ratio of MeOH and water in solution. The extraction is performed under stirring at room temperature (e.g., about 20° C. to about 25° C.) for about 30 minutes. After centrifugation, the solution is divided into two equal volumes. To the first volume part of the solution, the organic solvent is evaporated under vacuum at about 50° C. The remaining aqueous phase is freeze-dried to form Extract C.

The second volume part of the solution is treated with about 2 g of polyvinylpolypyrrolidone under stirring for about 30 minutes to trap polyphenols. The adsorbant material is removed by filtration, centrifugation or the like. The organic solvent is evaporated under vacuum at about 50° C. The remaining aqueous phase is freeze-dried to form Extract D.

Cytotoxic Effects of Chicory Extract

Experimental tests were conducted to determine non-cytotoxic doses of the chicory extract using MTT assay. Rat hepatocytes were treated with varying amounts of chicory extract B, the preparation of which was previously discussed. The test results indicated that cytotoxicity of chicory extracts was observed at about 200 micrograms/milliliter or more.

Western Blot Analysis

Whole protein extracts from cell cultures treated for 48 hours with chicory extracts (A-D) were resolved by SDS-PAGE on 10% gels (10 or 25 μg protein/lane for glutathione-S tranferase (“GST”) and heat shock protein (“HSP”) analyses respectively) using the discontinuous buffer system of Laemmli and transferred electrophoretically to nitrocellulose membranes. Blots were incubated for 1 hour in a solution of 5% dried milk in PBS containing 0.1% Tween to block protein binding sites. The blots were incubated with rabbit polyclonal antibodies raised against rat GST Ya, Yc, Yb1, Yb2, Yp ( BIOTRIN) and rat GST Yc2 (made available from Dr. J. Hayes of Dundee University). The antibody against rat GST Yc2 is a polyclonal antibody known to cross-react with other rat GST alpha subunits. See, Hayes J. D. et al., Biochemical :J, 279, 385-398, 1991; and Cavin C. et al., The coffee-specific diterpenes cafestol and kahweol protect against aflatoxin B1-induced genotoxicity trough a dual mechanism, Carcinogenesis 19, 1369-1375, 1998.

On Western blots, two bands are typically visible, with the upper and lower bands corresponding respectively to Yc1 and Yc2 subunits. The blots were also incubated with the rat polyclonal antibody raised against Heme-oxygenase-1 (HO-1) also called HSP 32 (stressgene, AMS). Blots were subsequently probed with a secondary antibody conjugated to horseradish peroxidase. The complex was visualized indirectly by chemiluminescence using an ECL western blotting detection kit (made available from AMERSHAM LIFE SCIENCE, England). Protein concentrations were measured using the method of Bradford which was standardized using BSA.

The test results indicated that Chicory Extract B which was made with ethyl acetate as previously discussed, increased the expression of some GSTs subunits (Yc2, Yb1, Yp) in rat hepatocytes as compared to controls and cells treated with Chicory Extracts A, C and D.

Enzymatic Assays

GST activities of cytosolic fractions were assayed as described in Habig W. G. et al, Glutathione-S-transferases: the first enzymatic step in mercapturic formation. Journal of Biological Chemistry 249, 7130-7139 (1974). 1-chloro-2,4-dinitrobenzene (CDNB) was used as a substrate to measure general GST activity in rat primary cultures treated with varying amounts of chicory extracts, namely Extracts A-D as previously discussed. The incubations were performed at 30° C.

Further, ethacrynic acid was used as a substrate to measure specific GST-P enzymatic activity in rat primary cultures treated with varying amounts of chicory extracts, namely Extracts A-D as previously discussed.

The test results indicated that Extract B resulted in an increased level of GST activity in rat hepatocytes as compared to controls and Extracts A, C and D.

Determination of Total Glutathione

Total glutathione level in cells was measured by enzymatic recycling as described in Gallagher E. P. et al., Glutathione, Oxidized glutathione and mixed disulfides in biological samples, In Methods in toxicology Vol. 1B, 349-366 (1994). Glutathione (GSH) is measured with a kinetic assay which utilizes the continuous glutathione reductase-catalyzed reduction of the sulfhydryl reagent 5,5′-dithiobis-2-nitrobenzoic acid (DTNB; Ellman's reagent) to the chromophoric product 2-nitro-5-thiobenzoic acid. Detection of the chromophore is monitored spectrophotometrically at 412 nm.

Rat primary hepatocytes were treated for 48 hours with varying concentrations (50, 100 and 200 μg/ml) of chicory Extract A (hexane), Chicory Extract B (ethyl acetate) and chicory Extract C (MetOH/H20). Cells were washed and resuspended in 125 microliters of 20 mM 5-SSA. Cellular GSH was then released by sonification and samples centrifuged at 10000 g for two minutes at room temperature. The supernatant containing free GSH was then used for the determination of total glutathione. Sample or GSH standard were mixed with 700 μl of 125 mM sodium phosphate containing 6.3 mM EDTA (pH 7.5), 100 μl of 6 mM DTNB and 20 μl of 20 mM NADPH. After equilibration of cuvettes to 25° C., 10 μl of GSSG reductase (50 U/ml) is added to measure the formation of 2-nitro-5-thiobenzoic acid at 412 nm. A sample blank lacking GSH is run separately and the resulting background formation of product formation is subtracted from the sample value prior to GSH quantification.

The test results indicated that Chicory Extract B resulted in increased cellular GSH levels in rat hepatocytes as compared to controls and Chicory Extracts A and C. Further, the test results indicated that the cellular levels of GSH increased with increased amounts of Chicory Extracts B.

Effect of Food Processing

Test samples were prepared to evaluate the effects food processing on the detoxification properties of the plant material constituent of the resultant food product. The test was conducted on a pet food that contained about 30% by weight of chicory made in accordance with an embodiment of the present invention. The pet food was then processed by extraction with ethyl acetate in accordance with an embodiment of the present invention. The resultant extract was added to rat hepatocyte cultures in varying amounts, namely 100 μg/ml, 200 μg/ml and 400 μg/ml. Western blot analysis was then conducted to determine the effects of the chicory extract on the GST activity of the hepatocyte cultures.

The test results indicated that the food processing procedure did not reduce and in fact enhanced the detoxification properties of the plant material, namely chicory. In this regard, an increased level of GST activity was measured with respect to the varying amounts of chicory extract added to the cultures. Further, the level of GST activity increased with increasing amounts of chicory extract.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

The invention is claimed as follows:

1. A nutritional composition capable of reducing a risk of cancer comprising a biologically effective amount of a plant material that includes a source of fermentable dietary fiber and a phytochemical agent capable of inducing enzyme activity in a mammal.

2. The nutritional composition of claim 1 wherein the plant material comprises an amount of at least 0.5%, on a dry weight basis.

3. The nutritional composition of claim 1 wherein the plant material is selected from the group consisting of chicory, Jerusalem artichoke, leek, asparagus extract thereof, pulp thereof, root thereof and combinations thereof.

4. The nutritional composition of claim 1 wherein the plant material comprises chicory selected from the group consisting of chicory extract, chicory root, chicory pulp and combinations thereof.

5. The nutritional composition of claim 1 wherein the source of fermentable dietary fiber comprises a prebiotic fiber selected from the group consisting of inulin, oligosaccharides, fructo oligosaccharides, soy oligosaccharides, and combinations thereof.

6. The nutritional composition of claim 1 wherein the phytochemical agent is selected from the group consisting of an antioxidant, cafestol, kahweol, catechins and combinations thereof.

7. The nutritional composition of claim 1 wherein the enzyme activity is conducted via glutathione-S-transferase.

8. The nutritional composition of claim 7 wherein the enzyme activity is capable of promoting detoxification in tissue of the mammal.

9. The nutritional composition of claim 7 wherein the enzyme activity is capable of stimulating an antioxidant defense in tissue of the mammal.

10. The nutritional composition of claim 1 wherein the nutritional composition is selected from the group consisting of a nutritional supplement, a nutritionally complete food product, a food preparation, a cereal product and a pet food.

11. A pet food product for reducing a risk of cancer comprising:
a starch matrix; and
a biologically effective amount of a plant material comprising a prebiotic fiber and a phytochemical agent capable of inducing enzyme activity in a mammal to reduce the risk of cancer.

12. The pet food of claim 11 wherein the plant material is selected from the group consisting of chicory root, Jerusalem artichoke, leek, asparagus, extract thereof, pulp thereof, root thereof and combinations thereof.

13. The pet food of claim 11 wherein the pet food comprises about 0.5% or more on a dry weight basis of the plant material.

14. The pet food product of claim 11 wherein the phytochemical agent is selected from the group consisting of an antioxidant, cafestol, kahweol, catechins and combinations thereof.

15. The pet food product of claim 11 wherein the enzyme activity is performed via glutathione-S-transferase to stimulate detoxification in tissue of the mammal.

16. The pet food of claim 11 wherein the enzyme activity is derived from glutathione-S-transferase to stimulate an antioxidant defense in tissue of the mammal.

17. The pet food of claim 11 wherein the plant material comprises chicory selected from the group consisting of chicory extract, chicory root, chicory pulp and combinations thereof.

18. A pet food product comprising a plant material including a source of fermentable dietary fiber and a phytochemical agent in an amount effective to reduce risk of cancer wherein the plant material is selected from the group consisting of chicory, Jerusalem artichoke, leek, asparagus, extract thereof, pulp thereof and combinations thereof.

19. The pet food product of claim 18 wherein the plant material is in an amount of at least 0.5%, on a dry weight basis.

20. The pet food product of claim 18 wherein the source of fermentable dietary fiber comprises a prebiotic fiber selected from the group consisting of inulin, oligosaccharides, fructo oligosaccharides, soy oligosaccharides, and combinations thereof.

21. The pet food product of claim 18 wherein the phytochemical agent is selected from the group consisting of an antioxidant, cafestol, kahweol, catechins and combinations thereof.

22. A method of preparing a nutritional food product capable of reducing a risk of cancer in a mammal, the process comprising the steps of:
providing a plant material;
processing the plant material to form a plant extract including a prebiotic fiber and a phytochemical agent capable of inducing enzyme activity in the mammal; and
processing the plant extract and one or more food ingredients to form the nutritional food product that includes at least 0.5% by weight of the plant extract.

23. The method of claim 22 wherein the plant material is selected from the group consisting of chicory, Jerusalem artichoke, leek, asparagus and combinations thereof.

24. The method of claim 22 wherein the plant extract is processed by defatting the plant material to form a first plant extract and subsequently processing the first plant extract with ethyl acetate via acid hydrolysis to form the plant extract.

25. The method of claim 22 wherein the enzyme activity is derived from glutathione-S-transferase.

26. A method of reducing a risk of cancer in a mammal at risk of cancer, the method comprising administering to the mammal a biologically effective amount of a nutritional composition that contains a plant material including a prebiotic fiber and a phytochemical agent.

27. The method of claim 26 wherein the nutritional composition contains at least 0.5% by weight of the plant material selected from the group consisting of chicory, Jerusalem artichoke, leek, asparagus, extract thereof, pulp thereof, root thereof and combinations thereof.

28. The method of claim 26 wherein the plant material comprises a plant extract derived from chicory.

29. The method of claim 28 wherein the nutritional composition contains about 0.5% to about 2% by weight of the plant material.

30. A method of increasing detoxification in tissue of a mammal, the method comprising administering to the mammal a biologically effective amount of a nutritional composition including a plant material that contains a prebiotic fiber and a phytochemical agent capable of inducing an enzymatic activity in the mammal.

31. The method of claim 30 wherein the nutritional composition comprises about 0.5% to about 2% by weight of the plant material selected from the group consisting of chicory, Jerusalem artichoke, leek, asparagus, extracts thereof, pulp thereof, root thereof and combinations thereof.

32. The method of claim 30 wherein the enzymatic activity is performed via glutathione-S-transferase.

33. A method of stimulating an antioxidant defense in tissue of a mammal, the method comprising administering to the mammal a biologically effective amount of a nutritional composition including a plant material that contains a prebiotic fiber and a phytochemical agent capable of inducing an enzymatic activity in the mammal.

34. The method of claim 33 wherein the nutritional composition comprises about 0.5% to about 2% by weight of the plant material selected from the group consisting of chicory, Jerusalem artichoke, leek, asparagus, extracts thereof, pulp thereof, root thereof and combinations thereof.

35. The method of claim 33 wherein the increased enzymatic activity results in an increased level of glutathione.
